(19) United States
(12) Patent Application Publication
(10) Pub. No.: US 2026/0363558 A1
(43) Pub. Date: Nov. 19, 2026

(54) SYSTEM AND METHOD OF MONITORING HEALTH OR SAFETY OF INDIVIDUALS AT DEFINED LOCATIONS

(71) Applicant: Vigilum Technologies Pty Ltd, Sydney (AU)

(72) Inventors: John Joseph Fenga, Sydney (AU); Steven Richard Lewis, Sydney (AU); Julius John Markos, Sydney (AU); Aleks Todorovic, Sydney (AU)

(21) Appl. No.: 19/148,255

(22) PCT Filed: Mar. 20, 2023
(86) PCT No.: PCT/AU2023/050196

§ 371 Date: Jul. 15, 2025

(30) Foreign Application Priority Data
Jan. 16, 2023 (AU) 2023900087

(51) Int. Cl. A61B 5/0205; A61B 5/00

(52) U.S. Cl. CPC A61B 5/0205; A61B 5/0024; A61B 5/0077; A61B 5/7445

(57) ABSTRACT

The present disclosure relates to systems and methods of monitoring health or safety of individuals.

### TECHNICAL FIELD

The present disclosure relates broadly to systems and methods of monitoring health or safety of individuals, such as workers, at multiple locations, such as worksites and workplaces.

### BACKGROUND

Operations in limited and/or confined spaces are inherently high-risk. Research suggests that a large proportion of limited and/or confined space injuries and fatalities involved first responders, who rushed in to help someone in a limited and/or confined space with little understanding of the conditions they would face. For example, asphyxiation due to lack of oxygen as well as the presence of dangerous gases, which can be spontaneously released during operations, can be fatal. With reductions in available safety crew, there is a need for a new and/or alternative way to monitor health or safety of individuals in high-risk operations, such as mining and industrial, to facilitate attention and/or quicken response of first responders.

Reference to any prior art in this document is not, and should not be taken as, an acknowledgement or any form or suggestion that the prior art forms part of the common general knowledge in any jurisdiction.

### SUMMARY OF DISCLOSURE

According to a first aspect of the present disclosure there is provided a system for monitoring health or safety of individuals located at multiple locations, the system including:
multiple capturing devices each configured to:
   capture, over time, vision associated with a corresponding one of the multiple locations; and
   provide visual data, generated based on the captured vision, via a data communications network;
multiple wearable or portable sensors each configured to:
   sense, over time, one or more health or safety metrics associated with a corresponding one of the individuals; and
   provide health or safety data, generated based on the one or more sensed health or safety metrics, via the data communications network; and
a data access device being remote from the multiple capturing devices and multiple sensors, the data access device being operatively coupled to:
   the data communications network for receiving the visual data and the safety data; and
   a display for synchronously displaying the visual data and the safety data.

According to a second aspect of the present disclosure there is provided a system for monitoring health or safety of individuals located in multiple locations, the system including:
a data access device being remote from multiple capturing devices and multiple wearable or portable sensors, the data access device being operatively coupled to a data communications network for receiving, and a display for synchronously displaying:
   visual data generated based on vision captured, over time, by multiple capturing devices, each associated with a corresponding one of the multiple locations, and provided via the data communications network; and
   health or safety data generated based on one or more health or safety metrics sensed, over time, by the multiple sensors, each associated with a corresponding one of the individuals, and provided via the data communications network.

According to a third aspect of the present disclosure there is provided a graphical user interface (GUI) on a data access device for monitoring health or safety of individuals located at multiple locations, the data access device being remote from multiple capturing devices and multiple wearable or portable sensors, the data access device being operatively coupled to a data communications network, the GUI including:
a first area for displaying visual data that is:
   generated based on vision captured, over time, by the multiple capturing devices each associated with a corresponding one of the multiple locations, and
   received via the data communications network; and a second area for displaying health or safety data that is:
      generated based on one or more health or safety metrics sensed, over time, by the multiple wearable or portable sensors each associated with a corresponding one of individuals, and
      received via the data communications network,
wherein the visual data and the health or safety data are synchronously displayed.

According to a fourth aspect of the present disclosure there is provided a method for monitoring health or safety of individuals located at multiple locations, the method including the steps of:
receiving, via a data communications network, visual data generated based on vision captured, over time, by multiple capturing devices each associated with a corresponding one of the multiple locations;
receiving, via the data communications network, health or safety data generated based on one or more health or safety metric sensed, over time, by multiple wearable or portable sensors each associated with a corresponding one of individuals; and
synchronously displaying, on a data access device being remote from the multiple capturing devices and the multiple sensors, the visual data and the safety data.

According to a fifth aspect of the present disclosure there is provided a computer-readable medium including a plurality of instructions which, when executed by a processor, cause the processor to perform the method in accordance with the fourth aspect.

According to a sixth aspect of the present disclosure there is provided a data access device for monitoring health or safety of individuals located at multiple locations, the data access device including:
one or more processors; and
memory which stores a plurality of instructions which, when executed by the one or more processors, cause the one or more processors to perform the method in accordance with the fourth aspect.

According to a seventh aspect of the present disclosure there is provided a sub-system for monitoring health or safety of individuals located at multiple location, the sub-system including:
multiple capturing devices each configured to:
   capture, over time, vision associated with a corresponding one of the multiple locations; and
   provide visual data, generated based on the captured vision, via a data communications network to a data access device operatively coupled to a display, the data access device being remote from the multiple capturing devices and multiple wearable or portable sensors, wherein the display is configured to synchronously display:
   the visual data; and
   health or safety data generated based on one or more health or safety metrics sensed, over time, by the multiple wearable or portable sensors each associated with a corresponding one of the individuals, and provided via the data communications network.

According to an eighth aspect of the present disclosure there is provided a sub-system for monitoring health or safety of individuals located at multiple locations, the sub-system including:
multiple wearable or portable sensors each configured to:
   sense, over time, one or more health or safety metrics associated with a corresponding one of the individuals; and
   provide health or safety data, generated based on the sensed one or more health or safety metrics, via a data communications network to a data access device operatively coupled to a display, the data access device being remote from multiple capturing devices and the multiple sensors,
wherein the display is configured to synchronously display:
   the safety data; and
   visual data generated based on vision captured, over time, the by multiple capturing devices each associated with a corresponding one of the individuals and provided via the data communications network.

Additional features are described in, and will be apparent from, the following Detailed Description and the figures.

### BRIEF DESCRIPTION OF DRAWINGS

In order to achieve a better understanding of the nature of the present disclosure, one or more embodiments of systems and methods for monitoring health or safety of individuals will now be described, by way of example only, with reference to the accompanying illustrations in which:

FIG. 1 illustrates an embodiment of a system for monitoring health or safety of individuals;

FIG. 2 illustrates an embodiment of a data access device and its graphical user interface (GUI) of the system of FIG. 1;

FIG. 3 illustrates a flow chart of an embodiment of a method performed by a data access device of FIG. 1;

FIG. 4 illustrates an example of a system for monitoring health or safety of individuals at a mining site;

FIG. 5 illustrates a series of graphical user interfaces of a data access device of FIG. 4; and

FIG. 6 a series of graphical user interfaces of a data access device of FIG. 4.

### DETAILED DESCRIPTION

The present disclosure relates broadly to systems and methods of monitoring health and safety of individuals and/or infrastructure in action at multiple locations. The individuals may engage in high-risk or vulnerable operations. The multiple locations may expose the individuals to harmful elements. In one example, the individuals may be workers in a constructions site, where asbestos or micro-fibrous materials may become airborne and inhalable. In another example, the individuals may be aircraft refuelling personnel in an airport refuelling station, at which any leakage of inflammable gas or liquid can create an explosive atmosphere. In yet another example, the individuals may be miners in a limited and/or confined space, in which depletion of oxygen may induce loss of consciousness. In still another example, the individuals may be patients or doctors in an operating theatre, in which disease-carrying pathogens may become airborne to cause infections. In a further example, the individuals may require decision support or control validation, for example due to limited jurisdictions or lack of authority to proceed with certain actions. In a still further example, the individuals or teams of individuals may be dispersed in large infrastructure, where the individuals or teams of individuals are physically too far apart for effective monitoring. The disclosed systems and methods facilitate the remote oversight, for example by monitoring personnel and/or an automatic monitoring and alert system, of such individuals' potential exposure to harmful elements in these multiple locations.

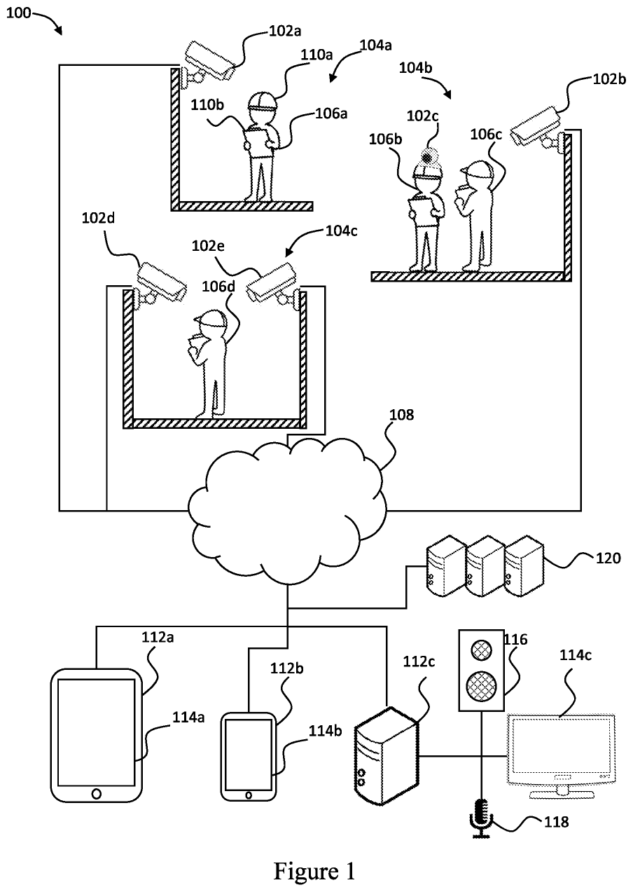

FIG. 1 illustrates an embodiment of a system 100 for monitoring health or safety of individuals in accordance with the present disclosure. The system 100 includes multiple capturing devices 102a, 102b, 102c, 102d and 102e (individually or collectively 102) each configured to capture, over time, vision associated with a corresponding one of multiple locations 104a, 104b and 104c (individually or collectively 104) and/or multiple individuals 106a, 106b, 106c and 106d (individually or collectively 106). The multiple capturing devices 102 are each further configured to provide visual data, generated based on the captured vision, via a data communications network 108. The data communications network 108 may include one or more of a mobile network, a satellite network, a Bluetooth network and a Wi-Fi network, and any of its interfaces.

One or more of the capturing devices (e.g. 102a, 102b, 102c and 102e) may be fixed. In one example, a fixed capturing device is a CCTV camera, in wired communication (e.g. via Ethernet) with the data communications network 108. In another example, the fixed capturing device is a webcam, in wireless communication (e.g. via Wi-Fi) with the data communications network 108. Alternatively or additionally, one or more of the capturing devices (e.g. 102d) may be movable. For example, a movable capturing device is an action camera, wearable by an individual and in wireless communication (e.g. via Bluetooth) with the individual's mobile device (e.g. mobile phone or tablet) and/or the data communications network 108.

The visual data may be in one of a number of digital formats, such as MP4 or MOV, using one of a number of compression or encoding protocols, such as H264 or H265. The visual data may be time-stamped.

Each location 104 may be associated with one individual (e.g. individual 106a at or near location 104a) or multiple individuals (e.g. individuals 106b and 106c at or near location 104b). Each location may be associated with one capturing device (e.g. capturing device 102a at or near location 104a) or multiple capturing devices (e.g. capturing devices 102d and 102e at or near location 104c).

The system 100 further includes multiple wearable or portable sensors (110a and 110b, individually or collectively 110) each configured to sense, over time, one or more health or safety metrics associated with a corresponding one of multiple individuals 106. The corresponding individual may, at any one time, be associated with a single location. Therefore, the one or more health or safety metrics associated with the corresponding individual may alternatively or additionally be associated with one or more of the multiple locations. Similarly, the captured vision associated with the corresponding location may alternatively or additionally be associated with one or more of the multiple individuals.

The multiple wearable or portable sensors 110 each provide health or safety data, generated based on the one or more sensed health or safety metrics, via the data communications network 108. In one example, the sensor is integrated with an individual's apparel, such as a hard hat 110a that has one or more built-in sensors. In another example, the sensor is a hand-carried sensor 110b. Examples of sensors include temperature sensors, humidity sensors, gas sensor, atmospheric sensor, particulate matter sensor, etc. Each location 104 may be associated with one or multiple sensors. In one embodiment, the health or safety data are generated via an Application Programming Interface (API) from Honeywell Safety Suite connected sensors.

The one or more health or safety metrics may include any one or more of the following: presence or level of environmental elements associated with one or more of the multiple locations, location(s) associated with one or more of the multiple individuals, biometric or vital signs such as heart rate associated with one or more of the multiple individuals, and distress signals associated with one or more of the multiple individuals. The health or safety data may be in the form of tiered categories, such as “high”, “medium” and “low” for oxygen levels. Alternatively or additionally, the health or safety data may be in the form of numeric values, such as concentration (e.g. 20%) for oxygen levels. The health or safety data may be time-stamped. Either or both of the visual data and health of safety data may be encrypted.

The system 100 further includes one or more data access devices (112a, 112b and 112c, individually or collectively 112) being remote from the multiple capturing devices 102 and multiple sensors 110. The one or more data access devices 112 are each operatively coupled to the data communications network 108 for receiving the visual data and the health or safety data. The one or more data access devices 112 are further each operatively coupled to a display 114a, 114b and 114c (individually or collectively 114) for synchronously displaying the visual data and the safety data. Each of the one or more data access devices 112 may be one of a tablet 112a, mobile phone 112b and a desktop/laptop computer 112c. In an embodiment, the display (e.g. display 114a) may be integrated within the data access device 112 (e.g. tablet 112a). In another embodiment, the display (e.g. display 114c) may be operatively coupled to the data access device (e.g. desktop computer 112c) via a wired or wireless connection.

In some embodiments, one or more of the capturing devices are each configured to capture audio, such as via a microphone, generate audio data based on the captured audio. The capturing devices 102 may further be configured to provide the generated audio data via the data communications network 108. The capturing devices may therefore generate both visual data and audio data. The visual data and audio data may be combined into a single data stream. Alternatively the visual data and audio data may be separately provided.

The visual data and the audio data may be provided, via the data communications network 108, to one or more of the data access devices 112. In this case, monitoring personnel has oversight over the visual data and audio data, and hence the one or more of the multiple individuals. Alternatively or additionally, the visual data and the audio data may be provided, via the data communications network 108, to a monitor and alert system, such as a backend server (not shown). In this case, the monitor and alert system may be configured to receive either or both of the visual data and the audio data for automatic monitoring. For example, the monitor and alert system may include a speech or sound processing engine for monitoring the audio data for any audio sign of distress, such as distress language (e.g. “emergency-emergency” or “mayday-mayday”), distress code (e.g. “code 112”), and certain sounds (e.g. sound of explosion). Alternatively or additionally, the monitor and alert system may include an image processing engine for monitoring the visual data for any visual signs of distress, such as an incidents (e.g. fire), unconscious individuals (e.g. a prolonged period of body non-movement and/or closed eyes), and unwell individuals (e.g. foaming at mouth). The monitor and alert system may be configured to generate one or more alerts, based on the monitored visual and/or audio data.

In some embodiments, the one or more of the data access devices 112 each further include an audio output device 116, such as a speaker, for rendering first audio data, generated based on first audio captured by a capturing device 102. The first audio data may be time-stamped. Based on time stamps, the first audio data may be rendered synchronously with the display of the visual data and the health or safety data. Alternatively or additionally, the one or more of the data access devices 112 each further include an audio input device 118, such as a microphone, for capturing second audio and generating second audio data based on the second audio. The second audio data may be provided to, via the data communications network 108, and rendered by, for example by a speaker, a capturing device 102. Accordingly, two-way voice communication between any data access device and any capture device may be enabled.

The one or more data access device 112 may be configured to receive the same set of data, such as the same visual data and/or the same health or safety data. That is, the visual data and/or the health or safety data may be broadcast to the one or more data access device 112. Alternatively, the one or more data access devices 112 may be configured to receive different sets of data. For example, data access device 112a is configured to receive only visual data, and data access device 112b is configured to receive only health or safety data. As another example, data access device 112a is configured to receive all visual data and all health or safety data associated with a first one of the locations, and data access device 112b is configured to receive all visual data and all health or safety data associated with a second, different from the first, one of the locations. That is, the visual data and/or the health or safety data may be unicast or multicast to the one or more data access devices 112. The one or more data access devices 112 may be password-protected. Where visual data and/or health of safety data is encrypted, the one or more data access devices 112 may be configured to receive a password to decrypt the encrypted data. Different passwords may allow different access levels, such as receipt of a different set of data.

In one embodiment, the one or more data access device 112 may be a third-party device that, by default, is configured with no data access. Upon configuration or authorisation with data access (e.g. using a valid password), the third-party device may be shared or granted access to a defined set of data. Sharing or granting of data access by third-party devices may facilitate inter-agency support. For example, where an incident has occurred at a location monitored by the system 100, a specialist team may be authorised (e.g. using a valid password) to access data stored by the system 100 (e.g. stored in the one or more storages devices 120) to assist with incident assessment.

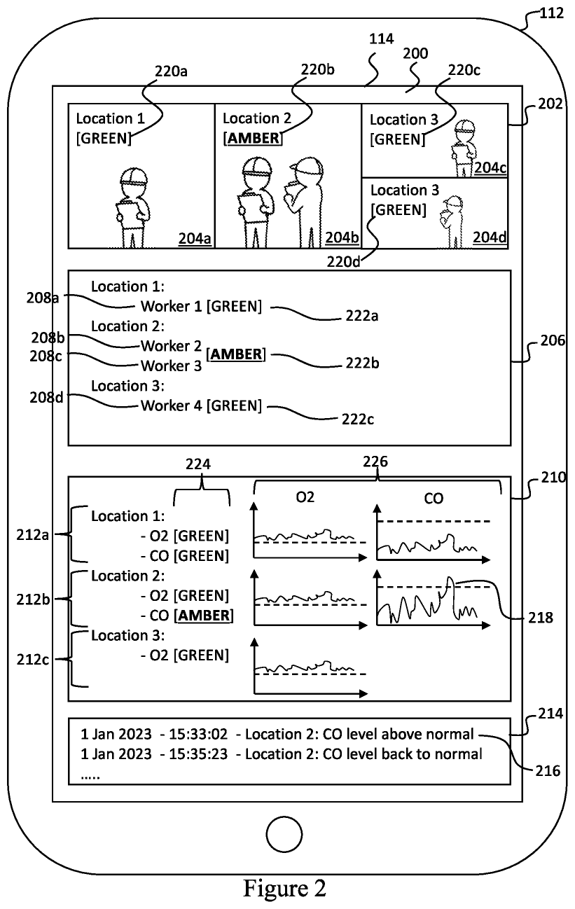

The one or more data access devices 112 may each be configured to provide a graphical user interface (GUI) on the display 114. FIG. 2 illustrates an example of a data access device 112 with a GUI 200. The GUI 200 may be a web-based interface, for example rendered by a web browser application.

The GUI 200 includes a first area 202 for displaying visual data 204a, 204b, 204c and 204d (individually or collectively 204) each associated with one or more corresponding locations 104. For example, visual data 204a is associated with location 104a, visual data 204b is associated with location 104b, and both visual data 204c and visual data 204d are associated with location 104c.

The displayed visual data and/or displayed health or safety data may be live-streamed, via the data communications network 108, at the one or more data access devices 112. That is, the broadcasted visual data and the health or safety data may displayed simultaneously at multiple data access devices 112. Live-steaming is facilitated by low network latency. The data communications network 108 may be configured to exhibit latency, from any one of the capturing devices 102 or any one of the sensor 110 to any one of the data access devices 112, of less than 500 ms, less than 200 ms or less than 100 ms. Alternatively the visual data 204 may be buffered or delayed, for example, by temporarily storing the visual data at the one or more data access devices 112 before displaying. Buffering mitigates network congestion, whereby either visual data or health or safety data lagging the other may be time-shifted for synchronous display. For example, streaming of visual data from capturing devices 102 is distributed via a cloud service Agora (http: titiwww.agora.ioti) allowing stream duplication and viewing at multiple data access devices 112.

The system 100 may further include one or more data storage devices 120, such as a server farm or a data centre, in data communication with the data communications network 108. Instead of or in addition to being provided to data access devices 112, the visual data and/or health or safety data may be provided to the one or more data storage devices 120 for storage. The stored data may be used for subsequent data analysis or historical reviewing.

The GUI 200 further includes a second area 206 for displaying individual data 208a, 208b, 208c and 208d (individually or collectively 208) associated with one or more corresponding locations 104. At any one time, each individual may be associated with a single location. The individual-location association may be facilitated by one of several localisation methods, such as global position system (GPS), received signal strength indicator (RSSI) and angle-of-arrival (AOA). For example, individual data 208a about Worker 1 is associated with location 104a, individual data 208b and individual data 208c about Workers 2 and 3 are associated with location 104b, and individual data 208d about Worker 4 is associated with location 104c.

The GUI 200 further includes a third area 210 for displaying health or safety data 212a, 212b and 212c (individually or collectively 212) associated with a corresponding location 104. While the health or safety data 212 is generated based on health or safety metrics sensed by sensors that are wearable or portable by the individuals 106, the health or safety data 212 can be made associated with a corresponding location 104 based on the association between an individual and a location at any one time.

The displayed health or safety data 212 may include one or more health or safety metrics. For example, health or safety data 212a about oxygen and carbon monoxide levels is associated with location 104a, health or safety data 212b about oxygen and carbon monoxide levels is associated with location 104b, and health or safety data 212b about oxygen level only is associated with location 104c. Time-stamped data may be displayed in a time series, such as in text form (e.g. tabulated oxygen level over time) or graphical form (e.g. a plot of oxygen level over time).

The correspondence of the multiple capturing devices to the multiple locations may be one-to-one, one-to-many, or many-to-one. Similarly, the correspondence of the multiple sensors to the multiple individuals may be one-to-one, one-to-many, or many-to-one. Further, each individual may be at one of multiple locations at any one time. Each location may therefore be associated with no, one, or more individuals. For example, as exemplified in FIGS. 1 and 2, Worker 1 is located at Location 1, and is monitored by a single capturing device 102a associated with Location 1. As another example, both Workers 2 and 3 are located at Location 2, and is monitored by two capturing devices 102b and 102c associated with Location 2. Worker 4 is located at Location 3, and is monitored by two capturing devices 102d and 102e. In addition, Worker 1 is further monitored by two sensors 110a and 110b worn or carried by Worker 1. Workers 2 and 3 each are similarly monitored by one or more sensors worn or carried by the worker and/or other workers. Worker 4 is monitored by one or more sensors worn or ported by the worker.

The GUI 200 is further configured to display a plurality of first alert indicators 220a, 220b, 220c and 220d (individually or collectively 220). The first alert indictors 220 are each associated with a corresponding location 104 or individual 106. The first alert indicators 220 are each indicative of an alert level based on the health or safety data 212. Alternatively or additionally, the first alert indicators 220 are each indicative of an alert level based on the monitored visual and/or audio data, for example monitored by the monitor and alert system. In one embodiment, the alert level may be indicated as one of several tiered levels, indicative of a progressively worse metric, respectively. The tiered levels may be in text format (e.g. GREEN, AMBER and RED) or colour-coded (e.g. colour green, colour amber and colour red). The health or safety data 212 may be associated with one or multiple health or safety metrics. For example, the first alert indicator 220a, indicating a GREEN alert level in this example, is indicative of the oxygen and CO levels of location 104a. The first alert indicator 220b, indicating an AMBER alert level in this example, is indicative of the oxygen and CO levels of location 104b. The first alert indicator 220c, indicating a GREEN alert level in this example, is indicative of only the oxygen level of location 104c. In this example, the AMBER alert level is based on the health or safety metric of CO level. The GREEN alert levels are based on other normal health or safety metrics. In case of multiple metrics, the first alert indicator 220 may be reflective of the worse or worst metric. Continuing the foregoing example, the first alert indicator 220b indicates an AMBER alert level due to a heightened level of CO, despite a normal level of oxygen (see further below).

The GUI 200 is further configured to display a plurality of second alert indicators 222a, 222b and 222c (individually or collectively 222). The second alert indictors 222 are each associated with a corresponding one or more of the multiple locations and/or multiple individuals. For example, the second alert indictor 222a is associated with location 104a and individual 106a. The second alert indictor 222b is associated with location 104b and individuals 106b and 106c. The second alert indictor 222c is associated with location 104c and individual 106d. Like the first alert indicators 220, the second alert indicators 222 are each indicative of an alert level based on the health or safety data 212 and/or the monitored visual and/or audio data, for example monitored by the monitor and alert system. Further, the second alert indictors 222 are each matched in appearance (e.g. by colour) with a corresponding one of the plurality of first alert indicators 220. For example, the AMBER alert level is indicated by both first alert indicator 220b and second alert indicator 222b. In this case, the AMBER alert level is based on the health or safety metric of CO level. The matching appearance facilitates identification of and quickens response to, for example by remote monitoring personnel, events, or incidents. Continuing from the foregoing example, by inspecting the first area 202 of the GUI 200, remote monitoring personnel may identify an AMBER alert level in Location 2. By further inspecting the second area 206 of the GUI 200, the remote monitoring personnel may further identify with relative ease that Workers 2 and 3, being located in Location 2 at the time, are exposed to potentially harmful elements.

The GUI 200 is further configured to display a plurality of third alert indicators 224. The third alert indicators 224 are each associated with a corresponding one of the multiple locations. For example, two indicators with GREEN alert level are each associated with Location 1. One indicator with GREEN alert level and one indicator with AMBER alert level are each associated with Location 2. Two indicators with GREEN alert level are each associated with Location 3. Like the first and second alert indicators 220 and 222, the third alert indicators 224 are each indicative of an alert level based on the health or safety data 212 and/or the monitored visual and/or audio data, for example monitored by the monitor and alert system. In this example, the health or safety data 212 are generated based on multiple health or safety metrics (here oxygen level and CO level). However, the first alert indicators 220 and second alert indicators 222 may only be indicative of the worse or worst metric. For example, each of the first alert indicator 220b and second alert indicator 222b indicate an AMBER alert level due to an abnormal level of CO, despite a normal level of oxygen.

Further, the third alert indictors 224 are each matched in appearance (e.g. by colour) with a corresponding one of the plurality of first alert indicators 220 or second alert indicators 222. For example, the AMBER alert level is indicated by all of first alert indicator 220b, second alert indicator 222b and one of the third alert indicators 224. In this case, the AMBER alert level is based on the health or safety metric of CO level. The matching appearance facilitates identification of the nature of the potential harm (e.g. abnormal level of CO) exposed to the individuals at the relevant location. For example, as illustrated in FIG. 2, the AMBER alert level indicated by the second alert indication 222b is not indicative of the nature of the potential harm. Instead, the third alert indicator 224 indicates that the AMBER alert level is associated with the CO level.

The GUI 200 is further configured to display the health or safety data in a time series, for example in the third area 210. Where the health or safety data is time-stamped, the times series may be displayed in text form (not down) or graphical form 226, such as one or more time plots. The one or more time plots facilitate identification of the time and/or duration of a historical or current potential harm. For example, the one or more time plots represent a, for example, heightened as opposed to depleted level of CO in Location 2 in the past.

The GUI 200 further includes a fourth area 214 for displaying a log of events or incidents. For example, the log may include multiple log items, such log item 216. Each log item may include date (e.g. 1 Jan. 2023), time (e.g. 15:33:02), duration of an alert (not shown), health or safety metric(s) on alert (e.g. CO levels), nature of potential harm (e.g. above normal). The log item may correspond to a feature of the displayed time series. For example, log item 216 corresponds to an overshoot 218 of CO level displayed in the third area 210.

Each data access device 112 may be one or more general purpose computing devices including one or more processors, such as microcontrollers or microprocessors, with computer readable memory storing instructions to cause the computing device or devices to perform the operations. Alternatively, each data access device 112 may each include one or more application specific devices configured to perform the operations described herein, such as one or more manufactured or configured programmable logic devices, such as application specific integrated circuits or field programmable gate arrays.

In the instance of a general purpose computing device, the data access device 112 may include, for example, a single computer processing device (e.g. a central processing unit, graphics processing unit, or other computational device), or may include a plurality of computer processing devices. The data access device 112 may also include a communications bus in data communication with one or more machine readable storage (memory or medium) devices which store instructions and/or data for controlling aspects of the operation of the data access device 112. The memory devices may include system memory (e.g. a BIOS), volatile memory (e.g. random access memory), and non-volatile memory (e.g. one or more hard disk or solid state drives to provide non-transient storage). The operations for health or safety monitoring are generally controlled by instructions in the non-volatile memory and/or the volatile memory.

In the instance of an application specific device, the instructions and/or data for controlling operation of the processing unit may be in whole or in part implemented by firmware or hardware elements, including configured logic gates. These elements may be integrated on a common substrate, for example as a system on a chip integrated circuit, or distributed across devices that are on separate substrates.

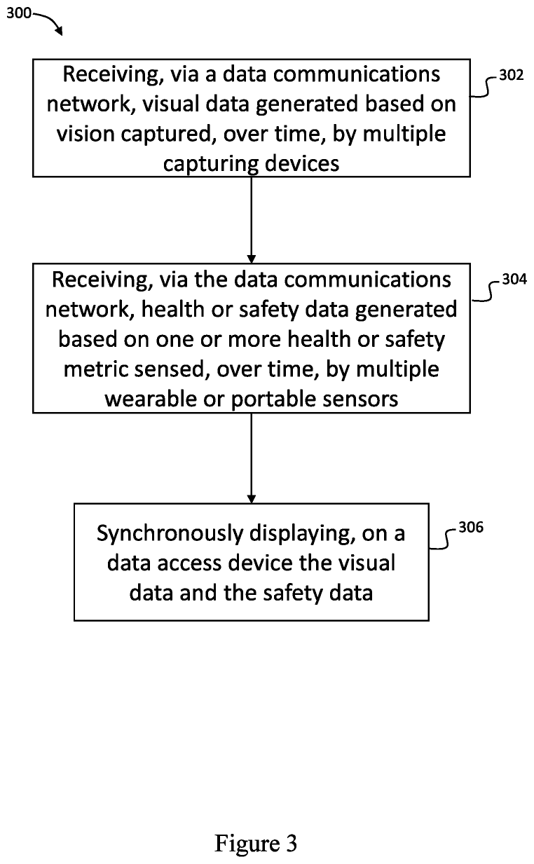

Whether the data access device 112 is a general purpose computing device or an application specific device does not affect the operations performed by the data access device 112. FIG. 3 illustrates a flow chart depicting an embodiment of a method 300 performed by the data access device 112, being remote from multiple capturing devices 102 and multiple wearable or portable sensors 110. Here, reference is made to the system and components described in relation to FIGS. 1 and 2. At step 302, the method 300 includes receiving, via the data communications network 108, visual data generated based on vision captured, over time, by the multiple capturing devices 102. At step 304, the method 300 further includes receiving, via the data communications network 108, health or safety data generated based on one or more health or safety metric sensed, over time, by the multiple wearable or portable sensors 110. As described above, the multiple capturing devices 102 are each associated with a corresponding one of multiple locations 104. The multiple sensors 110 are each associated with a corresponding one of multiple individuals 106. At step 306, the method 300 further includes synchronously displaying, on the data access device 112 the visual data and the safety data.

Components within the system 100 may be grouped into multiple sub-systems. For example, a sub-system may include the group of multiple capturing devices 102. Another sub-system may include the group of multiple wearable and portable sensors 110. Yet another subsystem may include the group of one or more data access device 112.

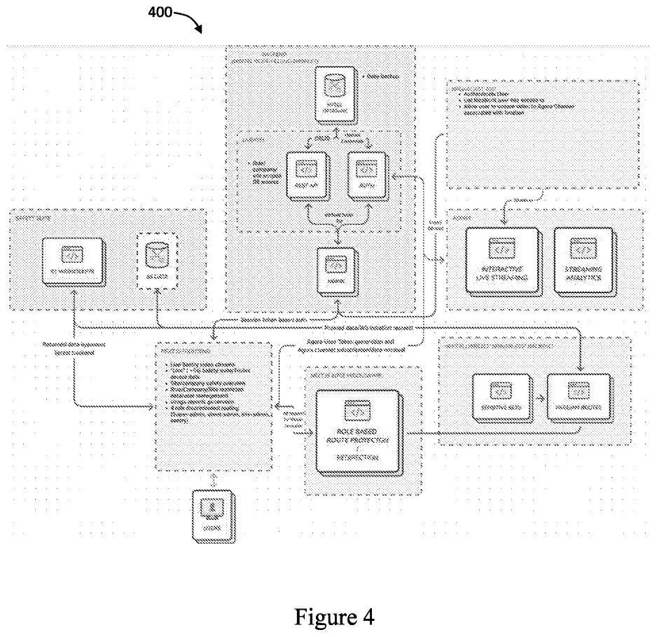

FIG. 4A illustrates an example of a system 400 for monitoring a mining site. The system 400 has 4 dedicated access roles: Sentry, Local, Remote and Administrative (Admin) to monitor the mining site. A further Super Administrator access role exists for high level systems administration. The system 400 supports live video streaming and visual indication of the workspace, combined with further data sourced from data streams (i.e., via API from Honeywell Safety Suite connected sensors). The system 400 provides constant visual monitoring and real time streaming from deployed capturing devices 102 and sensors 110.

The system 400 includes of a web-based sub-system and a mobile-based (e.g. Android-based) deployment application providing video streaming. The web-based sub-system can be accessed from any web connected device from any modern browser, including tablets and mobile phones.

The Architecture of the system 400 includes the following primary segments:
1. Front End Web Browser Based System (Next.js);
2. Server-less Scalable Back End Infrastructure Middleware (Vercel);
3. Database (MySQL), API and Authentication Host (NGINX, Laravel);
4. Device Data Sources (Honeywell Safety Suite);
5. Video Streaming Platform (Agora); and
6. Site Deployment Application (Android React Native).

### 1. Front End/PWA (Next.JS)

The Front End of the system 400 is built with Next JS, a React which features some useful backend capabilities like SSR/SSG, secured API routes, secured environmental variables, without maintaining a full backend framework. It is set up as a PWA (Progressive Web App) for the main purpose of achieving an app-like experience when using the sentry features on a tablet.

The Front end is where monitoring personnel directly accesses the web-based sub-system. For security, the system 400 is integrated with middleware mechanism using NEXT JS which supports role-based routing and auto redirection in the case that a user of the monitoring personnel is not authorised.

The Front End consists of 4 role discriminated UIs based on the following role classifications: Super Admin, Client Admin, Site Admin, and Sentry.

The primary actions a user of the monitoring personnel can take on the platform are: (a) Create & Manage sites, locations, workers, and sensors, (b) Usage Reports Generation, (c) User Management & Roles, (d) Live viewing of Sentry video streams and connected device data streams, (e) web based QR code or biometric (e.g. facial or fingerprint recognition) Clock in clock out of workers, (f) High Level Live Status overview of Video and Device Data Streams including warning and alarm levels, and (g) verification of workers' licenses due to, for example, expiry or revocation.

### 1.1 Super Admin Panel

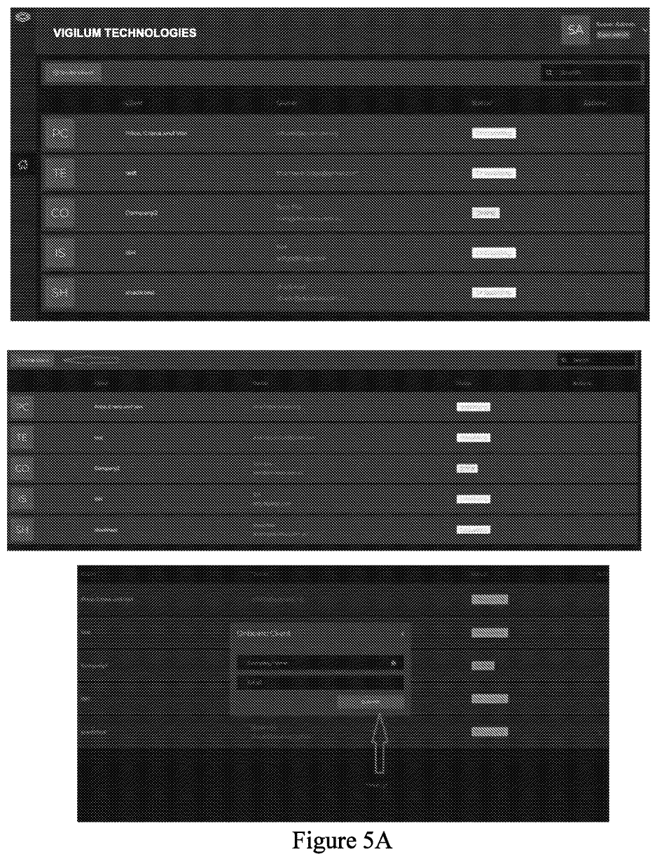

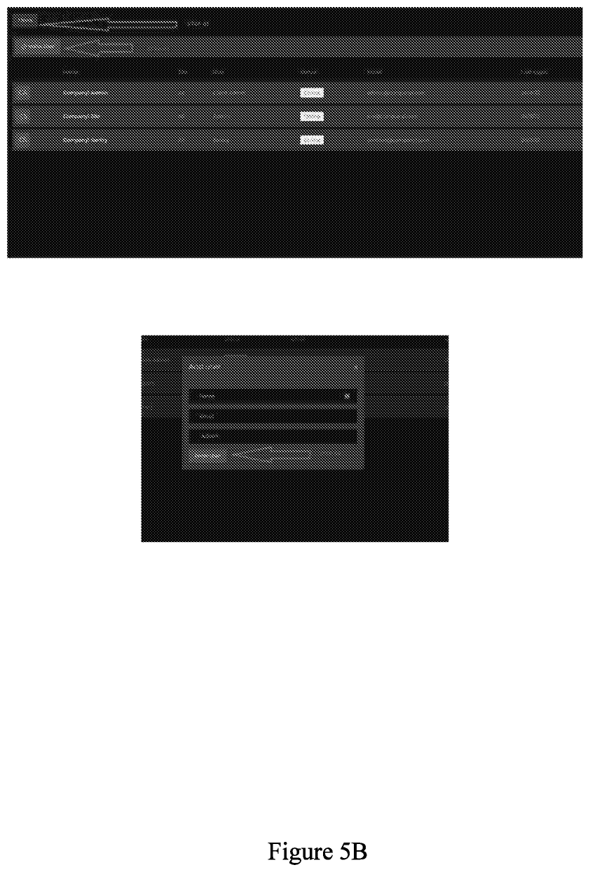

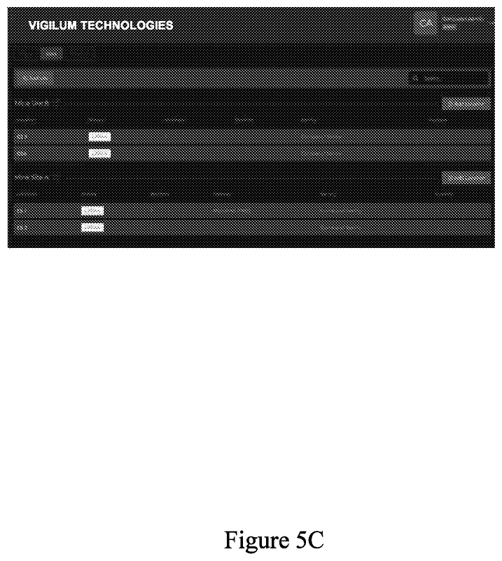

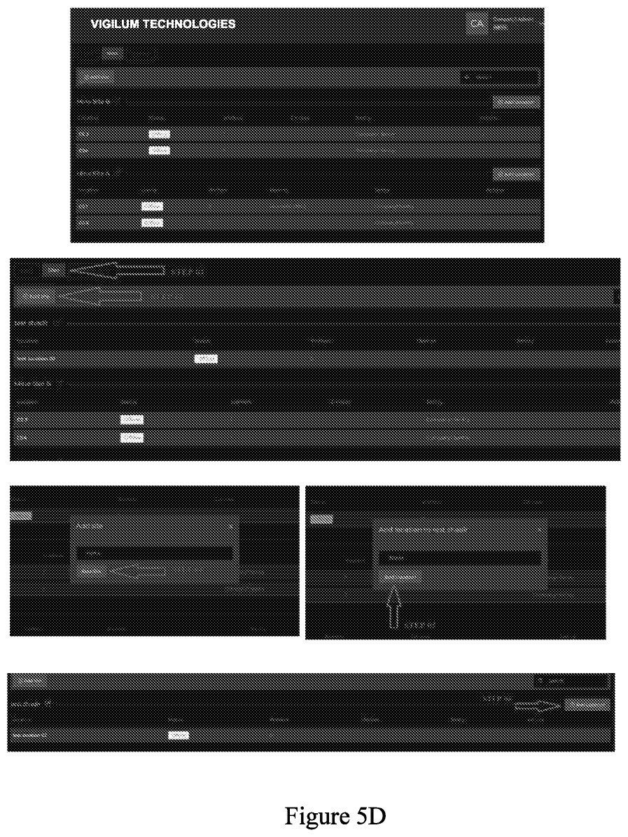

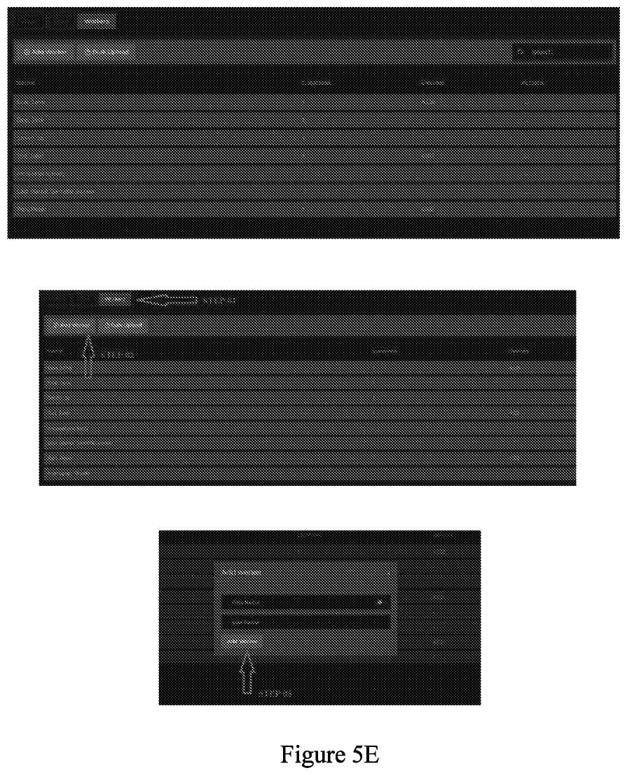

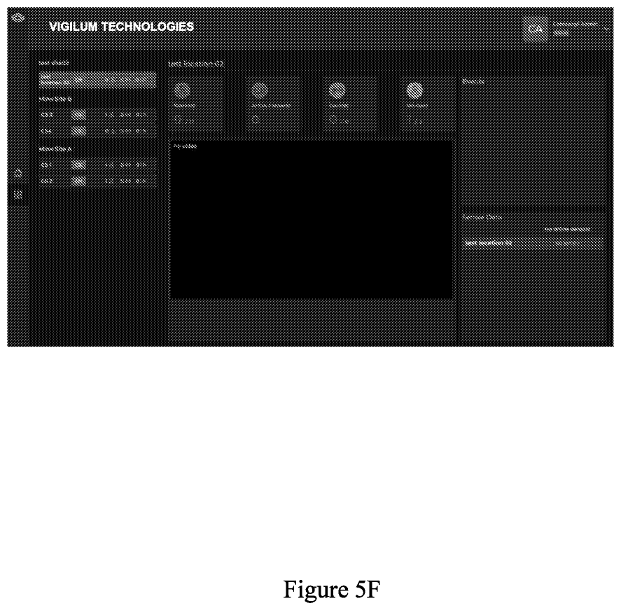

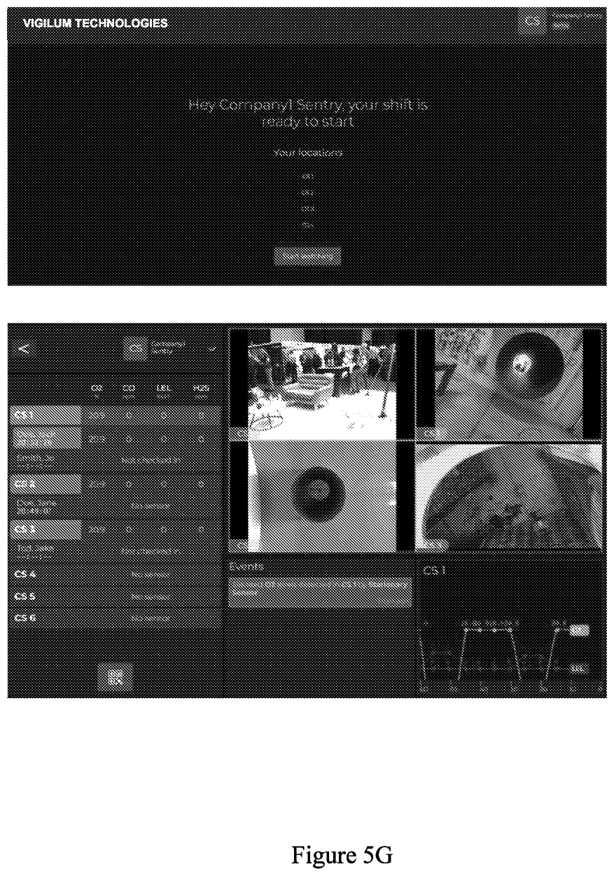

Referring to FIG. 5A, Super admin is the highest authority user type. Super Admin can create, manage, and update clients, client information, locations, capturing devices, & sensors. Super Admin can:
Invite or onboard clients to the system 400 and create the client account into the system 400;
View the status & information of the onboarding or online company;
Search for specific client(s);
Enter admin views for clients to impersonate roles as an Administrator from that client company.

Inviting clients to the system 400 includes the following steps:
Under Super Admin panel on the left corner click the “Invite client” button.
On board client dialog box will open up
Fill the Company name & the Email Address
Click the “submit” button to invite clients

This will trigger an email sign-up link to be sent to the inputted email, where user can add a password, as well as configure some other details

### 1.2. Admin Panel

The Admin Panel consist of these views:
7. User View
8. Sites View
9. Worker View
10. Control Panel View

### 1.2.1. Users View

Referring to FIG. 5B, Admin can:
invite users or remove
see user information such as name, avatar, site, role, status, email, last login & date they joined the platform

Inviting a user to the platform includes the following steps:
Go to the users view by clicking the user button in the top left of the screen
Click “invite User”
“Add User” dialog form will open, fill out users' information
Click “Invite user” to trigger the onboarding process. This will trigger an email invite with a magic link for the user to sign up with.

### 1.2.2. Sites View

Referring to FIG. 5C, this view contains information about sites and their locations and is the main interface for creating and editing sites or locations, assigning stationary sensors, assigning a sentry to a location, or assigning workers to a location.

This view acts as a quick summary of the following core location data:
Location name
Location status
Worker count
Assigned device name
Assigned sentry name

Adding a new site/location to the system 400 includes the following steps:
In admin panel first go to the Sites view port
Click “Add Site” Button
“Add Site” form will open
Fill location name text box in the form
Click “Add Site” button to add the site
After Creating site, admin must create locations for the site
On specific site which was created click “add location” button to create location
“Add location” dialog box will open
Fill the location name text box in the form
After filling the form click “add location” button to add the location to specific site

Referring to FIG. 5D, to assign a sentry, device, or worker include the following steps:
Click on the “Actions” ellipses on the location you wish to edit
Click from the dropdown options
A dialog form will appear to allow you to select relevant options
Click the submit button to update the IE system

### 1.2.2. Workers View

A worker is a person that will be assigned to work in a particular location and be observed by the Sentry. A worker will not have access to the system 400 but will automatically be assigned an ID and QR code when initiated on the system 400.

Referring to FIG. 5E, the interface contains the following core worker information:
Worker name
Number of current assigned locations
Name of current assigned device

Admins have the option of adding workers one by one or doing a bulk upload using a CSV file. As well as containing this information, the admin also has the ability to perform the following actions with the Actions dropdown to:
Configure a worker's location
Assign a device to a worker
Edit a worker's information
Get the workers QR code

### QR Code Scanning Mechanism

When a worker is created the system will generate QR code for them. This QR code is used to clock a worker in and out time at a particular location. After creating the QR code, the admin will share this code with the worker so that they can scan it when entering a controlled location. The Sentry for that location is responsible for scanning that code and confirming the location. Adding workers to the system includes the following steps:
In admin panel click “workers” tab
Click “Add Worker” Button
“Add Work” form will open up
Fill First & last name text box in the form
Click “Add Worker” button to add workers.

### 1.2.3. Control Centre View

Referring to FIG. 5F, this view shows a summary of all sites and locations, it contains information on number of assigned workers, number of active workers, as well as all camera and data streams; It is essentially sentry view but for all sites and locations.

### 1.3. Sentry Panel

Referring to FIG. 5G, the Sentry has the role of monitoring assigned locations, as well as clocking workers in and out of locations. The sentry view consists of 4 distinct areas:

In a first area: location and worker list with live sensor data (left)—This is the area that gives a full overview of all worker and sensor data for the locations that the sentry has been assigned to. The location or worker name can be one of four colours, indicating the hazard status of the location (green=safe, orange=alert, red=extreme risk, grey=no data), this colour corresponds to the colour of all related camera feeds to the right. The table will also indicate whether a sensor is online or not, or whether a worker is checked in (and if so, for how long). This table can handle a maximum of 5 sensor types over ALL locations. At the bottom of this section is a button to toggle the QR reader.

In a second area: responsive camera streams (top-right)—this area displays all the camera streams that are broadcast from the system 400, via the Agora platform. The number of videos on the screen at once is dynamic based on all the available streams for the location, there is no programmatically enforced limit to number of videos, however 9 is a recommended maximum, based on the available screen space. Each stream is labelled with its room name and has a coloured border to indicate the hazard level of the location.

In a third area: events register (bottom-centre)—a list of events (hazardous environmental reading) triggered by devices designated to the sentry's locations. There are two levels of event types: alert (orange), and extreme (red), and when the event has been cleared after a period of safe readings from the sensor that triggered the event, the event will turn green to indicate it has been resolved. This area also doubles as a QR reader for workers QR codes and is toggled by pressing the button at the bottom of area 1.

In a fourth area: live data graph (bottom-right)—This area shows a 60 second graphical representation of the data from a chosen device or sensor, this can be useful to identify rapid changes in gas concentrations for that device's location. To pick a device, click on its row in area 1. The active device will be highlighted.

### 2. Server-Less Backend and Edge Middleware

There are two more components to the Next. JS server-less front end infrastructure, the edge middleware and “server-less” back end. The edge middleware makes it extremely fast to perform rewrites are reroutes from anywhere in the world, it is currently being used to protect page and API routes by validating cookies on the edge network and rewriting to a login page if no valid cookies are found.

The server-less backend handles all SSR, SSG, and provides a convenient API system from the platforms URL origin. It may be used to proxy requests from the front end, to attach sensitive keys to requests to 3rd party APIs like Agora and Safety Suite.

### 3. Backend

In the system 400, the backend all runs within a Digital Ocean Cloud Droplet; a cloud-based virtual Linux (Ubuntu 20.04.5 LTS [GNU/Linux 5.4.0-125-generic x86_64]) server that allows for rapid processor, memory and storage scaling. Other benefits to DO Droplets include:
High level of default security
99.99% uptime
Redundancy measures like system wide backups and previous state restoration
Convenient visualisation tools and alerts to diagnose performance issues

To access the server, either a ssh handshake can be made with a device, or there is a virtual terminal that can be accessed through the droplet's web console.

The web server is a NGINX instance that runs as a “virtual host” for the Laravel API and auth server, allowing for future advance configuration and scaling.

Laravel is used as the backend framework, handling REST API, data schemas, user authentication, and email capabilities. Laravel includes security features such as data sanitation for API requests and rate limiting, amongst other security benefits. It is set up as a single tenant system with role, company, and site-based data protection ensuring all data is only available to those with authorisation. That is, a user can only fetch records if the user's role, company and site definition allow it. This approach is preferred over a multi-tenanted system as it removes the requirement for developers to develop new environments for every new client, and any performance disadvantages are negated through the scaling capabilities of the DO Droplet and NGINX.

A Single MySQL instance may handle all data, but can quickly be horizontally or vertically scaled as needed. All its data is captured in the Droplet's daily backup.

4. Safety Suite

Honeywell Safety Suite is a sub-system which provides application of safety, productivity for plant, site, remote and emergency responses by using devices and sensors. Safety suite manages mining site gas detection and monitors workers safety, from remote place. With safety suite module integration Sentry of the system 400 can maintain and track capturing devices and sensors so that they can reliably use to protect their workers and assets. Moreover, this module monitors the worker's exposure to gas in real time and sends data to the web-based sub-system.

5. Agora Interactive Live Streaming Platform

In the system 400, live video streaming of monitoring workers runs with Agora Real-Time voice & video engagement platform. Agora allows the system 400 to host large-scale video streaming events with real-time interactive live streaming API. Agora allows for fully customizable SDK & flexible APIs give developers control. Other benefits include:
HD Experience—High-definition videos apply up to 1080p cross-platform is mobile friendly with 1080p and 30 fps
Fast Channel Switching—Supports sub-second channel switching which will let IE to have seamless live video streaming experience.
Fast Rendering—Real-time Seamless video supports happen with fast frame rendering rate.
Adaptive Bitrate—Based on IE's hardware and network bandwidth smart encoder or decoder algorithm of agora delivers best resolution.
Efficiency—Agora's perception-based algorithm lets to reduces bandwidth needed and video rate without breaking the live video streaming quality.
Dual Video Live Streams

### 6. Broadcasting Mobile (e.g. Android) App

In system 440, the broadcasting mobile (e.g. Android) application consists of four viewpoints: Welcome page, Login page, Locations list page, and Streaming video channel page. The mobile application is configured for authentications of the users, show case lists of locations, allowing user to stream video using Agora channel associated with a specific location.

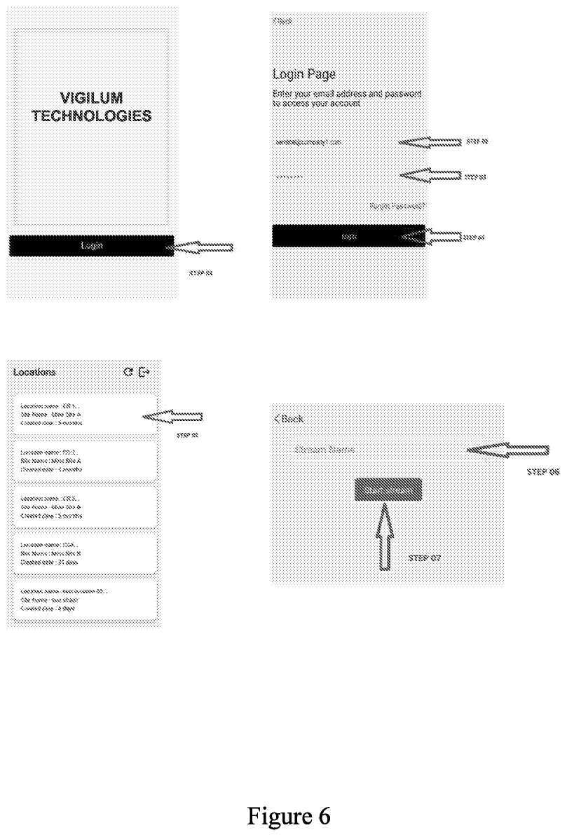

Referring to FIG. 6, the steps to access the mobile (e.g. Android) application includes:
In app go the Welcome page
Click “Login” button
Login Page will open up
Enter the Email Address & password and click the
“Login” Button
Locations list page will open up
Click on of the location list to Stream
Streaming page will open
Enter the Stream name in text box and click “Start
Stream” to being the streaming

Now that embodiments of the present disclosure have been described it should be understood that the computer-implemented method and system for monitoring health or safety have at least the following advantages:
1. Where oversight is conducted by monitoring personnel, the ratio of monitoring personnel to individuals being monitored may be one-to-many, such as any ratio between one-to-two and one-to-ten, to reduce human resource requirements;
2. Where alert indicators are matched in appearance between multiple areas of a GUI, the matched appearance facilitates identification of, and/or quickens response to, events or incidents by monitoring personnel;
3. The visual and/or health or safety data quality review, quality assessment or maintenance checks of on-site installations by remote skilled observer and/or appraiser;
4. The visual and/or health or safety data facilitates to verify conditions and compliance with regulations, for example, in real-time; and
5. Use of data access devices facilitates training at off-site locations.

Those skilled in the art will appreciate that the disclosure described herein is susceptible to variations and modifications other than those specifically described. All such variations and modifications are to be considered within the scope of the present disclosure the nature of which is to be determined from the foregoing description.

1. A system for monitoring health or safety of individuals located at multiple locations, the system including:
multiple capturing devices each configured to:
capture, over time, vision associated with a corresponding one of the multiple locations; and
provide visual data, generated based on the captured vision, via a data communications network;
multiple wearable or portable sensors each configured to:
sense, over time, one or more health or safety metrics associated with a corresponding one of the individuals; and
provide health or safety data, generated based on the one or more sensed health or safety metrics, via the data communications network; and
a data access device being remote from the multiple capturing devices and multiple sensors, the data access device being operatively coupled to:
the data communications network for receiving the visual data and the safety data; and
a display for synchronously displaying the visual data and the safety data.

2. The system of claim 1, wherein the data access device is further configured to display, on the display, a plurality of first alert indicators, each indicating an alert level based on the health or safety data and each being associated with a corresponding one of the multiple locations and/or the individuals.

3. The system of claim 2, wherein the data access device is further configured to display, on the display, a plurality of second alert indicators, each being matched in appearance with a corresponding one of the plurality of first alert indicators and each being associated with captured vision associated with a corresponding one of the multiple locations and/or individuals.

4. The system of claim 1, wherein the visual data and the health or safety data are time-stamped for synchronization.

5. The system of claim 1, wherein
the multiple capturing devices are further configured to capture, over time, first sound associated with the corresponding one of the multiple locations, and provide first audio data, generated based on the captured sound, via the data communications network; and
the access device is further operatively coupled to a first audio output device for rendering the first audio data synchronously with display of the visual data and the health or safety data.

6. (canceled)

7. (canceled)

8. The system of claim 1, wherein the data communications network is configured to exhibit data latency, between any one of the multiple capturing devices or any one of the multiple sensors and the data access device, of 500 ms or less.

9-12. (canceled)

13. The system of claim 5, wherein:
the data access device is further configured to capture second sound and provide second audio data, different from the first audio data, generated based on the captured second sound; and
one or more of the multiple capturing devices are each configured to receive the second audio data, via the data communications network, and each further include a second audio output device, different from the second audio output device, for rendering the second audio data.

14-27. (canceled)

28. A graphical user interface (GUI) on a data access device for monitoring health or safety of individuals located at multiple locations, the data access device being remote from multiple capturing devices and multiple wearable or portable sensors, the data access device being operatively coupled to a data communications network, the GUI including:
a first area for displaying visual data that is:
generated based on vision captured, over time, by the multiple capturing devices each associated with a corresponding one of the multiple locations, and
received via the data communications network; and
a second area for displaying health or safety data that is:
generated based on one or more health or safety metrics sensed, over time, by the multiple wearable or portable sensors each associated with a corresponding one of individuals, and
received via the data communications network,
wherein the visual data and the health or safety data are synchronously displayed.

29. The GUI of claim 28, wherein the data access device is further configured to display, on the display, a plurality of first alert indicators, each indicating an alert level based on the health or safety data and each being associated with a corresponding one of the multiple locations and/or the individuals.

30. The GUI of claim 29, wherein the data access device is further configured to display, on the display, a plurality of second alert indicators, each being matched in appearance with a corresponding one of the plurality of first alert indicators and each being associated with captured vision associated with a corresponding one of the multiple locations and/or individuals.

31. The GUI of claim 28, wherein the visual data and the health or safety data are time-stamped for synchronization.

32. The GUI of claim 28, wherein
the multiple capturing devices are further configured to capture, over time, first sound associated with the corresponding one of the multiple locations, and provide first audio data, generated based on the captured sound, via the data communications network; and
the access device is further operatively coupled to a first audio output device for rendering the first audio data synchronously with display of the visual data and the health or safety data.

33. (canceled)

34. (canceled)

35. The GUI of claim 28, wherein the data communications network is configured to exhibit data latency, between any one of the multiple capturing devices or any one of the multiple sensors and the data access device, of 500 ms or less.

36-39. (canceled)

40. The GUI of claim 28, wherein:
the data access device is further configured to capture second sound and provide second audio data, different from the first audio data, generated based on the captured second sound; and
one or more of the multiple capturing devices are each configured to receive the second audio data, via the data communications network, and each further include a second audio output device, different from the first audio output device, for rendering the second audio data.

41. A computer-readable medium including a plurality of instructions which, when executed by a processor, cause the processor to perform the steps of:
receiving, via a data communications network, visual data generated based on vision captured, over time, by multiple capturing devices each associated with a corresponding one of the multiple locations;
receiving, via the data communications network, health or safety data generated based on one or more health or safety metric sensed, over time, by multiple wearable or portable sensors each associated with a corresponding one of individuals; and
synchronously displaying, on a data access device being remote from the multiple capturing devices and the multiple sensors, the visual data and the safety data.

42-54. (canceled)

55. The computer-readable medium of claim 41, wherein the data access device is further configured to display, on the display, a plurality of first alert indicators, each indicating an alert level based on the health or safety data and each being associated with a corresponding one of the multiple locations and/or the individuals.

56. The computer-readable medium of claim 55, wherein the data access device is further configured to display, on the display, a plurality of second alert indicators, different from the plurality of first alert indicators, the plurality of second alert indicators each being matched in appearance with a corresponding one of the plurality of first alert indicators and each being associated with captured vision associated with a corresponding one of the multiple locations and/or individuals.

57. The computer-readable medium of claim 41, wherein the visual data and the health or safety data are time-stamped for synchronization.

58. The computer-readable medium of claim 41, wherein
the multiple capturing devices are further configured to capture, over time, first sound associated with the corresponding one of the multiple locations, and provide first audio data, generated based on the captured sound, via the data communications network; and
the access device is further operatively coupled to a first audio output device for rendering the first audio data synchronously with display of the visual data and the health or safety data.

59-65. (canceled)

66. The computer-readable medium of claim 58, wherein:
the data access device is further configured to capture second sound and provide second audio data, different from the first audio data, generated based on the captured second sound; and
one or more of the multiple capturing devices are each configured to receive the second audio data, via the data communications network, and each further include a second audio output device, different from the first audio output device, for rendering the second audio data.

67-105. (canceled)
